(19) United States
(12) Patent Application Publication
(10) Pub. No.: US 2016/0213724 A1
(43) Pub. Date: Jul. 28, 2016

(54) AQUEOUS PLANT EXTRACTS

(71) Applicants: Rafael GALLART GRAU, BARCELONA (ES); María Asunción GALLART GRAU, Montornès Del Vallès (ES)

(72) Inventors: Salvador CAÑIGUERAL FOLCARÀ, BARCELONA (ES); Simona SCIOTTO, BARCELONA (ES); Roser VILA CASANOVAS, BARCELONA (ES); Ester RISCO RODRIGUEZ, BARCELONA (ES)

(73) Assignees: Rafael GALLART GRAU, BARCELONA (ES); María Asunción GALLART GRAU, MONTORNÈ DEL VALLÈS (ES)

(21) Appl. No.: 15/026,936

(22) PCT Filed: Oct. 3, 2014
(86) PCT No.: PCT/EP2014/071244

§ 371 Date: Apr. 1, 2016

(30) Foreign Application Priority Data
Oct. 4, 2013 (ES) P 201331465

(51) Int. Cl. A61K 36/28; A61Q 19/00; A61K 8/97

(52) U.S. Cl. CPC A61K 36/28; A61K 8/97; A61Q 19/00; A61K 2236/331

(57) ABSTRACT

The present invention relates to a process comprising: (a) suspending the plant in water; (b) macerating the suspension obtained in step (a); and (c) separating the resulting liquid obtained in step (b); wherein in any step prior to step (c) the plant is submitted to a quantity of laser energy equal to or less than 3 J/cm², wherein: the exposure time is comprised from 1 to 30 minutes; laser wavelength is comprised from 815 to 1,400 nm; the laser power is comprised from 20 mw to 60 W; the distance from the laser source to the plant is comprised from 10 to 150 cm; and the amount of plant exposed to the laser is comprised from 0.1 to 1 Kg/m², to an extract obtainable by said process, and compositions containing them; and the use thereof in cosmetics and therapy.

The present invention relates to a process for preparing aqueous extracts of plants and to the aqueous extracts thus obtained, topical compositions comprising the same and the application thereof in cosmetics and in the treatment of inflammatory skin diseases.

### STATE OF THE ART

Inflammatory skin diseases refer to the majority of the most common rashes such as, for example, eczema, dermatitis and psoriasis. These diseases are usually chronic and mainly transcurs through the following symptoms: itching, redness, swelling, dryness and desquamation.

In particular, psoriasis is a common skin disease characterized by epidermal hyperplasia caused by aberrant hyperproliferation of the keratinocytes and by the presence of red scaly plaques. In this chronic disease, lesions are typically subject to remissions and exacerbations. There are several patterns; although plaque psoriasis is the most common, there is also psoriasis guttata, having lesions in the shape of water drops spread around the trunk and extremities, and pustular psoriasis, which are usually located on the palms of the hands and soles of the feet. The most common inflammatory lesions vary from discrete erythematous papules to plaques covered with silvery scales.

The symptoms of psoriasis need to be treated to control the severity and propagation frequency of the disease. There are different systemic and topical pharmacological treatments comprising retinoids, anti-inflammatory agents such as corticoids, and moisturizing agents of natural origin as aloe vera or rosehip oil.

However, in spite of the extensive scientific literature and numerous treatment strategies, there is still no effective treatment for psoriasis without side effects.

Plants are an important source of active ingredients used in medicine. Processes used to extract active ingredients from plants are very important with regard not only to the yields of the compounds to be obtained, but also to the chemical nature of the compounds forming part of the extracts.

Processes that are usually used are based on extracting from the crushed plant with water or steam, with organic solvents, or with mixtures of water and organic solvents such as alcohols. These processes may also include a maceration step. Lastly, separation of the aqueous extract of the solid phase may be carried out by means of conventional processes such as decantation, centrifugation or filtration.

Optionally, different additional chemical or physical treatments which can modify the composition of active ingredients in the extract may also been applied.

In particular, Calendula officinalis L., also known as calendula or buttercup, is an herbaceous, aromatic, glandular, annual to perennial plant, which is widely cultivated as an ornamental species. The extracts of flower buds and/or petals of calendula have frequently been used in popular medicine as immunostimulator, anti-tumour, anti-inflammatory, antibacterial and antioxidant.

Different processes have been described in the state of the art for the preparation of extracts of calendula. Some of these processes comprise laser irradiation of the plant.

The international patent application WO200241908 discloses a process for extracting calendula, previously irradiated with laser light having a wavelength comprised between 150 to 810 nm, as well as the obtained extract and compositions comprising it. In particular, it is experimentally disclosed the irradiation with a laser having a 250 nm wavelength and 20 watts of power. It also discloses the activity of these extracts as stimulators of growth and lymphocytic activity, which is useful for treating immunosuppressive diseases, among which psoriasis is included. It specifically discloses that laser treatment of the plant may catalyse reactions between certain compounds of plants and/or facilitate the extraction of certain compounds and, thereby, the presence of said compounds in the extract may modify their pharmacological activity.

The international patent application WO200242335 discloses a group of polysaccharides isolated from a calendula extract, the plant having been previously irradiated with laser light having a wavelength comprised between 150 and 810 nm. In particular, it is experimentally disclosed the irradiation with a laser whose wavelength is 250 nm and a power of 20 watts. It also discloses the activity of said polysaccharides as stimulators of the growth and lymphocytic activity, which is useful for treating immunosuppressive diseases, among which psoriasis is included.

Finally, Garrido et al. discloses the preparation of a calendula flower extract wherein said flowers have been previously laser irradiated at 650 nm. It also discloses the activity of said extracts on lymphocyte stimulation and as a cytotoxic anti-tumour. It also mentions that the increase in biological activity is caused by laser irradiation treatment, which may induce conformational changes by stimulating or degrading different molecules in the extract. (cf. Garrido et al. “A new extract of the plant Calendula officinalis produces a dual in vitro effect: cytotoxic anti-tumour activity and lymphocyte activation”, BMC cancer, 2006, Vol. 6, pp. 119).

Therefore, there is still a need to find new and more efficient treatments, without side effects, for inflammatory skin diseases such as psoriasis.

### EXPLANATION OF THE INVENTION

The inventors have developed a process for preparing plant extracts that comprises submitting the plant to a quantity of laser energy (also called fluence) as defined in the invention.

Thus, the first aspect of the present invention relates to a process for preparing an extract of one or more plants comprising: (a) suspending the plant in water; (b) macerating the suspension obtained in step (a); and (c) separating the resulting liquid obtained in step (b); wherein in any step prior to step (c) the plant is submitted to a quantity of laser energy equal to or less than 3 J/cm², wherein: the exposure time is comprised from 1 to 30 minutes; the laser wavelength is comprised from 815 to 1,400 nm; the laser power is comprised from 20 mw to 60 W; the distance from the laser source to the plant is comprised from 10 to 150 cm; and the amount of plant exposed to the laser is comprised from 0.1 to 1 Kg/m².

The inventors have found that the process of the invention, which comprises submitting the plant to the quantity of laser energy defined in the invention, makes it possible to obtain an extract from said plant having active ingredients content that is different from the extracts defined in the state of the art. In particular, it is thought that carrying out the extraction process with a plant submitted to the conditions of the present invention makes it possible to modify quantitative and qualitative composition of the polysaccharides, among other active components, present in the extract.

Thus, the extract of the present invention is characterized by having higher anti-inflammatory activity than the extracts described in the state of the art, being useful for preventing and/or symptomatically treating inflammatory skin diseases, including psoriasis (cf. Example 2).

The term “extract” of a plant is used in its conventional meaning referring to preparations of liquid, semi-solid or solid consistency, which may be concentrated or not concentrated, obtained from plant material by extracting the active ingredients from the plant by suitable means. Such active ingredients may be obtained from different parts of the plants. The suitable means for extracting the active ingredients include, for example, the use of water, organic solvents, microwaves or extraction with supercritical fluids. The active ingredients sometimes are directly incorporated in foods or in pharmaceutical or cosmetic compositions in a variety of forms, including as a pure or semi-pure component, the solid, semi-solid or liquid extract, or as solid material consisting of plant. Plant extracts contain not only one but several constituents, many of them active. Often, the beneficial effect is derived from the combination of many of these compounds, even in cases where a particular compound is the one responsible for most of the activity.

The term “plant”, as used in the present invention, refers to a unit or various units of the same species; or various units of different species; or a part or several parts of one or several plants such as, for example, stems, leaves or flowers.

The term “macerate”, as used in the present invention, refers to the act of immersing a solid substance (plant) in a liquid for a period of time to extract the soluble parts thereof.

In an embodiment of the invention, the process of the invention is carried out on a plant belonging to the families selected from the group consisting of Asteraceae, Rosaceae, Lamiaceae, Santalaceae, Crucifrae, Labiatae, Equisetaceae, Saxifraganceae, Compositae, Araliceae, Umbeliferae and mixtures thereof; preferably, the plant belongs to the genus selected from the group consisting of Calendula, Agrimonia, Lamium, Viscum, and mixtures thereof.

In an embodiment of the invention, the process is carried out on a plant belonging to the species selected from the group consisting of Mentha x piperita L., Pimpinella anisum L., Eleutherococcus senticosus (Rupr. & Maxim.) Maxim., Echinacea purpurea (L.) Moench, Echinacea angustifolia DC:, Symphytum officinale L., Equisetum arvense L., Calendula officinalis L., Agrimonia eupatoria L., Lepidium latifolium L., Lamium album L. and mixtures thereof.

In a preferred embodiment, the specie is Calendula officinalis; more preferably the flowers of Calendula officinalis are used.

In an embodiment, the process for preparing a plant extract of the present invention further comprises a prior step of plant decontamination; preferably the decontamination step is carried out by treating with water.

In another embodiment, the process for preparing a plant extract of the present invention further comprises a prior step of crushing or shredding the plant; said crushing or shredding is carried out manually or alternatively by using a suitable crushing or shredding machine.

In step (a) of the process of the invention, the plant is suspended in water. Any distilled water, deionized water or drinking water may be used, preferably those which are compliant with the requirements for “water for extraction” established in the European Pharmacopoeia. In an embodiment, suspension step (a) is carried out when the weight/volume ratio between the weight of the plant and the water volume is comprised between 1:1 and 1:25; preferably with a ratio comprised between 1:5 to 1:15; more preferably, the ratio is 1:10.

Then, the suspension obtained in step (a) is macerated (step (b)) unstirring for a period of time comprised from 1 to 200 hours. In an alternative embodiment, step (b) is carried out for a period of time comprised from 1 to 3 hours. In another alternative embodiment, step (b) is carried out for a period of time comprised from 100 to 200 hours. Optionally, maceration step (b) may also be carried with agitation of the mixture. In an embodiment of the invention, this agitation is carried out periodically such as, for example, daily.

In an embodiment, step (b) is carried out at a temperature comprised from 1 to 90° C.; preferably step (b) is carried out at a temperature comprised from 3 to 70° C. In an alternative embodiment, step (b) is carried out at a temperature comprised from 50 to 70° C. In another alternative embodiment, step (b) is carried out at a temperature comprised from 3 to 10° C.

In an embodiment of the invention, step (b) is carried out at a temperature comprised from 50 to 70° C. for a period of time comprised between 1 to 3 hours. In an alternative embodiment, step (b) is carried out at a temperature comprised from 3 to 10° C. for a period of time comprised from 100 to 200 hours.

Then, the supernatant of the suspension obtained in step (b) is separated from the solid phase. In an embodiment, the separation step (c) of the process of the invention may be carried out by pressing the solid phase to facilitate the separation. In another embodiment of the invention, step (c) may be carried out by decanting or decanting followed by filtering. In an embodiment of the invention, step (c) is carried out by decanting followed by filtering, wherein the filtering step is carried out under pressure; preferably under low pressure (vacuum).

The process of the invention comprises submitting the plant at any step prior to step (c) to a quantity of laser energy equal to or less than 3 J/cm², wherein: the exposure time is comprised from 1 to 30 minutes; the laser wavelength is comprised from 815 to 1,400 nm; the laser power is comprised from 20 mW to 60 W; the distance from the laser source to the plant is comprised from 10 to 150 cm; and the amount of plant exposed to the laser is comprised from 0.1 to 1 Kg/m².

In an embodiment, the quantity of laser energy to which the plant is submitted is comprised between 0.3 to 3 J/cm².

In another embodiment, the exposure time to the laser is comprised from 1 to 15 minutes.

In another embodiment, the laser wavelength is comprised from 820 to 1,000 nm; preferably, from 830 to 980 nm.

In another embodiment, the laser power is comprised from 50 mw to 20 w.

In an embodiment, the distance from the laser source to the plant is comprised from 10 to 150 cm; preferably, the distance is comprised from 20 to 100 cm; more preferably, the distance is comprised from 50 to 70 cm.

To carry out the process of the invention, the plant is exposed to laser radiation such that all or the majority of the surface of the plant is irradiated. This is achieved either by moving a laser generator or a set of various generators along the surface of the plant, or by passing the plant on a conveyor belt along a set of various laser generators. In an embodiment, the amount of plant exposed to the laser is comprised from 0.2 to 0.5 Kg/m². Preferably, the amount of plant exposed to the laser is comprised from 0.3 to 0.4 Kg/m².

In an embodiment of the invention, the width of the laser at the focal point is comprised from 1 to 10 mm.

In an embodiment of the invention, the energy is applied over the plant in a step prior to step (a), wherein the plant may be crushed, whole, dry, or wet.

Alternatively, in another embodiment, energy is applied over the plant during maceration step (b). The plant may be crushed, shredded or whole; preferably crushed or shredded.

In an embodiment, the process of the invention further comprises after step (c) an additional step to obtain a dry extract, wherein the additional step is selected from:

Step (d), which comprises drying the aqueous extract obtained in step (c);

Step (e), which comprises:
(i) Precipitating the aqueous extract obtained in step (c) by adding an organic solvent, to obtain a precipitate and a supernatant; and
(ii) Drying the precipitate obtained in step (i); and

Step (f), which comprises:
(i) Carrying out one or more extractions of the aqueous extract obtained in step (c) with one or more organic solvents, and separating the aqueous phase and organic phase; and
(ii) Drying the aqueous phase obtained in step (i);

The steps of drying the aqueous extracts may be carried out by means of any method that is known in the state of the art. In an embodiment, in the process for preparing a dry extract previously described, the steps of drying the aqueous extract of the invention are carried out by a method selected from the group consisting of concentration under low pressure, lyophilisation or a combination thereof; preferably the drying step is carried out by lyophilisation.

In an embodiment, the process for preparing a dry extract of the invention wherein the organic solvent is selected from the group consisting of (C₂-C₆) ketone and (C₁-C₃) alcohol; preferably the solvent is alcohol (C₁-C₃) alcohol. In a preferred embodiment, the solvent used in the process for preparing the precipitated dry extract of the present invention is ethanol. In an alternative embodiment, the solvent used in the process for preparing the precipitated dry extract of the invention is acetone.

Another aspect of the invention relates to the extract obtainable by the process as defined in the present invention. In an embodiment, the extract obtainable by the process of the invention is an aqueous extract. In another embodiment, the extract obtainable by the process of the invention is a dry extract. The extracts obtainable by the process of the invention exhibit anti-inflammatory activity that is unexpectedly higher that the extracts obtained by means of the processes described in the state of the art. For the purpose of the invention, the terms “obtainable”, “obtained” and equivalent expressions are used interchangeably and, in any case, the phrase “obtainable” encompasses the term “obtained”.

The extract obtainable by the process of the invention may be in the form of topical pharmaceutical or cosmetic composition. Thus, another aspect of the invention relates to a topical pharmaceutical or cosmetic composition comprising an effective amount of the extract as defined in the present invention, together with one or more appropriate topical pharmaceutically or cosmetically acceptable excipients or carriers

An “effective amount” of the combination refers to the amount of extract that provides a therapeutic or cosmetic effect after its application.

The term “pharmaceutically acceptable” refers to the excipients or carriers appropriate for use in pharmaceutical technology, for the preparation of compositions for medical use.

The terms “cosmetically acceptable” or “dermatologically acceptable” have the same meaning, may be used interchangeably in the present document, and refer to excipients or carriers that are appropriate for use in contact with human skin without inappropriate allergic response, instability, incompatibility, or toxicity among others.

In an embodiment, the topical composition is a pharmaceutical composition comprising an effective amount of the extract previously defined together with one or more appropriate topical pharmaceutically acceptable excipients or carriers.

In another embodiment, the topical composition is a cosmetic composition comprising an effective amount of the extract previously defined together with one or more appropriate topical cosmetically acceptable excipients or carriers.

The topical pharmaceutical or cosmetic compositions defined above comprise excipients or carriers that are appropriate for topical administration, which may be pharmaceutical or cosmetic excipients and include, but are not limited to, skin barrier recovery agent, a hydrating agent, an emollient, an emulsifier, a thickener, a humectant, a pH-regulating agent, an antioxidant, a preservative agent, a vehicle or mixtures thereof. The excipients or carriers used have affinity with the skin, are well tolerated and stable, and are used in an appropriate amount to provide the desired consistency and ease of application.

Additionally, the compositions of the present invention may contain other ingredients such as, for example, fragrances, colorants, and other components known in the prior state of the art that are useful for topical formulations.

The topical compositions of the invention may be formulated in several ways including, but not limited to, solutions, creams, mousses, lotions, gels, ointments, pastes, shampoos, bath gel, body cleansers or facial cleansers.

The topical composition used is preferably formulated in form of an emulsion. An emulsion is a dispersed system comprising at least two immiscible phases, one phase dispersed in the other in form of drops. The emulsifier agents mentioned above are included to improve stability. When the dispersed phase is water and the dispersion medium is oil, the emulsion is called water-in-oil emulsion (w/o). When the oil is the one dispersed as drops in the aqueous phase, the emulsion is called oil-in-water (o/w). Other kinds of emulsion known in the state of the art are multiple emulsions, such as water-in-oil-in water emulsions (w/o/w), GELTRAP emulsions, where the internal aqueous phase is gelified and is covered by the oil phase, and SWOP emulsions, also known as inverse emulsions. Preferably, the emulsions used are oil-in-water emulsions. Preferably, the emulsions to be used in the present invention are compatible with creams and lotions.

The topical compositions of the present invention may be prepared by means of processes well known in the art. Suitable excipients and/or carriers, and the amounts thereof, may be easily determined by the person skilled in the art according to the kind of formulations to be prepared.

The pharmaceutical composition of the invention may be used for topical application on the skin for the prophylaxis and/or treatment of an inflammatory skin disease or condition as shown in the results of Example 2. Thus, another aspect of the present invention is the use of the topical pharmaceutical composition as defined in the present invention for preparing a medicament for use thereof in the prophylaxis and/or symptomatic treatment of an inflammatory skin disease or condition. This aspect may also be formulated as a topical pharmaceutical composition as defined above, for use thereof in the prophylaxis and/or treatment of an inflammatory skin disease or condition. It also relates to a method for the prophylaxis and/or treatment of a disease or condition of an inflammatory skin disease or condition comprising administering an effective amount of the extract defined above to a mammal in need of such treatment, together with one or more appropriate topical pharmaceutically acceptable excipients or carriers.

In an embodiment of the invention, the topical pharmaceutical composition of the invention may be used for topical application on the skin for the prophylaxis and/or symptomatic treatment of an inflammatory skin disease or condition.

The term “symptomatic treatment” refers to the medical strategy that comprises limiting the severity of symptoms instead of treating the underlying cause or ethiology of the disease.

In an embodiment of the invention, the inflammatory skin diseases and conditions are selected from the group consisting of psoriasis, cellulitis, allergic contact dermatitis, phototoxic and photoallergic dermatitis, erythema multiforme, erythema nodosum, solar erythema, folliculitis, bullous pemphigoid, cutaneous sarcoidosis, urticaria and leukocytoclastic vasculitis. In a preferred embodiment, the inflammatory skin disease or condition is psoriasis.

The cosmetic composition of the invention may be used for skin care. Thus, another aspect of the present invention is the use of the topical cosmetic composition as defined above for skin care. In a preferred embodiment, the cosmetic compositions of the present invention are used for skin care, wherein skin care comprises relieving at least one of the following symptoms: roughness, desquamation, itching, redness, swelling, dryness and pruritus of the skin. The topical cosmetic composition of the present invention is designed to be applied to the body to improve its appearance or beauty, or protect the skin. Therefore, the cosmetic compositions defined in the invention are used for a non-medical application. In an embodiment, the topical cosmetic composition of the present invention is used as moisturizer or emollient agent.

Throughout the description and claims the word “comprises” and its variants are not intended to exclude other technical characteristics, additives, components or steps. In addition, the word “comprises” includes the case “consists of”. For those skilled in the art, other objects, advantages and characteristics of the invention may be deduced from both the description and the practical use of the invention. The following examples and drawings are provided by way of illustration, and are not meant to limit the present invention. In addition, the present invention encompasses all the possible combinations of particular and preferred embodiments herein indicated.

### EXAMPLES

### Example 1

### Preparation of Dry Calendula officinalis Extract

A. Laser Light Radiation

300 g of shredded Calendula officinalis flowers were spread on a surface to have 0.3 Kg of plant per m². Subsequently, said plant was exposed for 5 minutes to a laser having a wavelength of 830 nm, and a power of 50 mw, which provided an energy of 1.5 J/cm², where the distance between the laser source and the plant was 50 cm.

B. Preparation of the Aqueous Extract 300 g of the irradiated Calendula officinalis flowers obtained in section A were shredded and suspended in 3 L of distilled water, and were macerated for 7 days at a temperature of 4 ° C. with daily manual agitation.

After this time, the resulting suspension was centrifuged at 4,000 rpm, for 10 minutes. Subsequently, the liquid was separated from the suspension by decanting and filtering under low pressure. The liquid phases obtained were pooled and filtered to obtain 1,950 ml of the liquid aqueous extract 1.

C. Preparation of the Dry Aqueous Extract

C1. By Lyophilisation

The aqueous extract obtained in step B was concentrated by evaporation at low pressure (vacuum) and the concentrate obtained was dried by lyophilisation to create a dry extract 1 having a yield of 25% by weight, with respect to the plant extracted.

C2. Fractionating by Precipitation 600 ml of the aqueous extract obtained in step B was concentrated by vacuum evaporation to 200 ml, and aqueous solution of 96% ethanol (800 ml) was slowly added to the concentrate obtained at 3-6° C. Subsequently, the mixture obtained was maintained unstirred at 5° C. for 12 h to obtain a precipitate. The precipitate thus obtained was separated from the supernatant by centrifugation at 2,000 rpm for 10 minutes and then was decanted.

The resulting precipitate was re-dissolved in water (60 ml) and submitted to a second precipitation by adding an aqueous solution of 96% ethanol (200 ml) at 3-6° C. Subsequently, the mixture obtained was maintained unstirred at 5° C. for 12 h to obtain a precipitate. The precipitate thus obtained was separated from the supernatant by centrifugation at 2,000 rpm for 10 minutes and then was decanted.

The supernatant obtained in the second precipitation was mixed with the supernatant from the first precipitation and then was concentrated by vacuum evaporation followed by lyophilisation to obtain a dry extract 2 (16.6% yield).

The precipitate obtained was concentrated by vacuum evaporation, and then lyophilized to obtain a dry extract 3 (5.1% yield).

C3. Fractioning by Liquid Extraction

600 ml of the aqueous extract obtained in step B were extracted by partition with 200 ml of dichlomethane. Two additional washes were carried out with 200 ml each time. The organic phases obtained in the three extractions were mixed and, subsequently, were concentrated by vacuum evaporation, to obtain a dry extract 4 (0.2% yield).

The resulting aqueous phases from the three extractions were combined and 200 ml of n-butanol were added. Four additional washes were carried out with 200 ml of n-butanol each time. The organic phases obtained were mixed and subsequently were concentrated by vacuum evaporation, followed by lyophilisation to obtain a dry extract 5 (1.9% yield).

The organic phases obtained were mixed and subsequently were concentrated by vacuum evaporation, followed by lyophilisation to create a dry extract 6 (18.1% yield).

### EXAMPLE 2

### Study of the Effectiveness and Tolerance of the Liquid Aqueous Extract 1 of Calendula officinalis Flower for Psoriasis

A. Design of the Study

The trial was carried out as a randomized, single-centre, simple blinded, placebo-controlled study, randomly distributed, in 30 patients having diagnostic criteria of mild and stable psoriasis, who stated their willingness to participate in the research after have being informed.

B. Volunteers

Testing was carried out in 30 patients whose age was comprised between 18 and 70 years, 16 of whom were included in the placebo group and 14 in the treated group. Patients should be willing and able to understand all the requirements of the study and meet them, and must comply with the washout of topical and systemic treatments within the past two weeks.

C. Test Substance

A composition in the form of a cream-gel was used (Batch No. 120101), comprising the liquid aqueous extract 1 from the Calendula officinalis flower obtained as described in Example 1, up to step B, packaged in white tubes of 100 to 110 ml for the test composition, as well as a placebo composition in the form of a cream-gel (Batch No. 120102), comprising only the excipients, packaged in white tubes of 100 to 110 ml.

All the applications were topically administered twice a day in the areas affected by psoriasis for 4 weeks (the period of time that period V2 lasted).

D. Test Process

The trial was performed for a period of time of 8 weeks from 2 Nov. 2011 to 3 Aug. 2012, and was constituted by 4 stages:

1. Screening visit (V1): the patients who met all inclusion criteria to participate were included in the trial.

2. Washout period: the patients enrolled in the trial discontinued the use of banned products and/or treatments for two weeks. Patients avoided using any other treatment for psoriasis throughout the whole study.

3. Treatment period (V2): the patients applied the test composition or placebo composition indicated in section (C) for 4 weeks. During this period, patients were visited every 2 weeks (V3 and V4) to evaluate the effectiveness and tolerance.

4. Follow-up period (V5): At least 2 weeks after the last application (V4), patients returned to the centre for the follow-up visit and to undergo the end-of-study evaluations.

E. Target Parameters

E.1. General Considerations

PASI Scores (“Psoriasis Area and Severity Index”)

Objective method that has been used since 1978 in patients having psoriasis, for clinical evaluation of therapeutic response in clinical trials.

It is obtained by combining in a mathematical formula the value placed on four parameters: erythema, infiltration, scales and affected area. This value is calculated by the visual inspection of the four main areas of the body, which are head, trunk, upper extremities and lower extremities.

The observer estimates the percentage of skin that seems affected by the disease (psoriasis) in each area and assigns scores based on the following scale of 0-6:

0=no participation

1=10%

2=10-30%

3=31-50%

4=51-70%

5=71-90%

6=91-100%

PASI score is calculated according the following formula:

PASI=0.1×(erythema+infiltration+scales)×area[head]+0.3×(erythema+infiltration+scales)×area[trunk]+0.2×(erythema+infiltration+scales)×area[upper extremities]+0.4×(erythema+infiltration+scales)×area[lower extremities].

The Severity of Psoriasis

Mild psoriasis: When psoriasis covers less than 2% of the body, it is considered mild. Generally, people with mild cases develop isolated patches of psoriasis on the knees, elbows, scalp, hands and feet.

Moderate psoriasis: When psoriasis covers from 2% to 10% of the body, it is considered a moderate case. Psoriasis can occur on the arms, legs, torso, scalp and other areas.

Severe Psoriasis: When psoriasis covers more than 10% of the body, it is considered severe. Large areas of skin may be covered with psoriasis plaques or pustules, or widespread erythrodermic psoriasis can cause severe desquamation of the skin.

PGIC Rating (“Patient Global Impression of Change”)

The patients rated the overall progress of their psoriasis on a scale of 0-7.

1=Great improvement

2=Significant improvement

3=Minimal improvement

4=No change

5=Minimal worsening

6=Significant worsening

7=Great worsening

The PGIC is based on CGIC (see below) and must be answered in control visits V3 and V4.

CGIC Rating (“Clinician Global Impression of Change”)

The physician will rate the change in the state of the subject as compared to the beginning of the study on a scale of 0-7:

1=Great improvement

2=Significant improvement

3=Minimal improvement

4=No change

5=Minimal worsening

6=Significant worsening

7=Great worsening

The CGIC must be answered in control visits V3 and V4.

E.2. Effectiveness of the Treatment

In order to investigate the effectiveness of the treatment in patients having mild psoriasis, said analysis was carried out with the entire set, which consisted of all those randomized subjects who have completed the study and have at least carried out at least two PASI scores (before and after separating the two groups into treated and placebo), and the set comprising all patients from the entire set of analysis who have finished the maintenance period and have not deviated from the protocol.

In this study the following parameters were analysed:
Changes in PASI scores between the beginning of the treatment period (V2) and the control points (V3 and V4);
The progression of the cutaneous lesions having a central core surrounded by concentric rings of paleness and redness (also called “dartboard”, “iris” or “target”) between the beginning of the treatment period (V2) and the control points (V3 and V4), determined by the decrease of the erythema, infiltration and desquamation by means of localization and evaluation with a 4-point VAS scale from 0=absence to 3=maximum;
The progression of psoriasis by PGIC;
The progression of psoriasis by CGIC; and
The progression of the symptoms of the pruritus plaques/sting/itching, redness, irritation and dryness. Patients must evaluate the intensity of the symptoms using a VAS scale comprised between 0 (absence) and 10 (maximum).

E.3. Tolerance to Treatment

In order to investigate the tolerance to the treatment in patients having mild psoriasis, said analysis was carried out in the tolerance population, that is all the randomized patients those received at least one application of the cosmetic product of the study.

In this study the following parameters were analysed:
Pruritus/sting/itching after exposure to the cosmetic product and
Compliance with treatment.

F. Results

PASI

The results of the clinical evaluation of the therapeutic response in the clinical trials of the present invention are summarized in Table 1.

TABLE 1

| | PASI | | | |
|---|---|---|---|---|
| | V1 | At 14 Days | At 24 Days | V5 |
| Placebo | 2.8 | 4 | 2 | 2 |
| Liquid aqueous extract 1 | 2.6 | 2 | 1 | 1.6 |
| EX. 1 | | | | |

The results in Table 1 show that at the end of the treatment period the group treated with the composition of the present invention exhibits a significantly higher reduction in the PASI values versus the no/placebo group. The PASI reduction in the treated group was 2.8 to 1 versus a 2.6 to 2 reduction in placebo group.

PGIC

The assessment of the patient's state both in the placebo group and the group treated with the liquid aqueous extract 1 from the Calendula officinalis flower of Example 1 show that there is a significant difference in the assessment of both groups. It must be considered that the placebo group V5 was only formed by 31% of the patients who finished the trial.

CGIC

The assessment of the patient's state both in the placebo group and the group treated with the composition of example 1 according the physician is detailed in Tables 2 and 3.

TABLE 2

| | CGIC | | | | | | | |
|---|---|---|---|---|---|---|---|---|
| | V1 | | V3 | | V4 | | V5 | |
| | | Liquid | | Liquid | | Liquid | | Liquid |
| | | aqueous | | aqueous | | aqueous | | aqueous |
| | | extract 1 | | extract 1 | | extract 1 | | extract 1 |
| Parameters | Placebo | EX. 1 | Placebo | EX. 1 | Placebo | EX. 1 | Placebo^{(a)} | EX. 1^{(b)} |
| Pruritus | 0.5 | 2 | 3 | 2 | 3 | 0 | 1 | 0 |
| Irritation | 2 | 2.5 | 4 | 2 | 3 | 1 | 1 | 0.5 |
| Redness | 4.5 | 4.5 | 5 | 2 | 4 | 1 | 1 | 0.5 |
| Dryness | 5 | 6 | 6 | 3 | 5 | 2 | 3.5 | 3 |

^{(a)}Results observed in individuals that completed the treatment (31%)
^{(a)}Results observed in individuals that completed the treatment (71%)

TABLE 3

| | CGIC | | | | | | | |
|---|---|---|---|---|---|---|---|---|
| | V1 | | V3 | | V4 | | V5 | |
| | | Liquid | | Liquid | | Liquid | | Liquid |
| | | aqueous | | aqueous | | aqueous | | aqueous |
| | | extract 1 | | extract 1 | | extract 1 | | extract 1 |
| Parameters | Placebo | EX. 1 | Placebo | EX. 1 | Placebo | EX. 1 | Placebo^{(a)} | EX. 1^{(b)} |
| Erythema | 1 | 1.5 | 1 | 1 | 1 | 1 | 0.5 | 0.5 |
| Infiltration | 1 | 1.5 | 1 | 1 | 1 | 1 | 0.5 | 0.5 |
| Desquamation | 1.5 | 2 | 2 | 1 | 2 | 1 | 1 | 0.5 |

^{(a)}Results observed in individuals that finished the treatment (31%)
^{(a)}Results observed in individuals that completed the treatment (71%)

The results shown in Tables 2 and 3 shows the reduction of the signs and symptoms of psoriasis in the affected areas treated with the composition comprising the extracts of the present invention. Furthermore, adverse effects associated with the application of the composition of the present invention were not observed.

However, a marked deterioration of the signs and symptoms of the affected areas was observed in the placebo group as shown in the placebo group V3 and V4. This deterioration in the state of these patients caused 69% of the patients in this group to leave the study. Thus, the results shown in Tables 2 and 3 for the placebo group V5 only refer to the 31% of patients in the placebo group that finished the treatment.

Lastly, from the results observed in Tables 2 and 3 it is apparent that the percentage of compliance with the treatment with the compositions of the present invention is very high, specifically 71%. This may be caused by the improvements observed in the signs and symptoms of the disease during the treatment.

To conclude, the results obtained in the study of the effectiveness and tolerance described in the present invention show an improvement in the patients treated with the composition comprising the extract of the present invention, as compared to the placebo group. Furthermore, the composition of the present invention is very well tolerated by patients because it does not have any adverse effects and facilitates, in this way, compliance with the treatment.

1. A process for preparing aqueous extracts of plants comprising:
(a) Suspending the plant in water;
(b) Macerating the suspension obtained in part (a); and
(c) Separating the resulting liquid obtained in part (b);
wherein in any part prior to part (c) the plant is submitted to a quantity of laser energy equal to or less than 3 J/cm², wherein:
the exposure time is comprised from 1 to 30 minutes;
the laser wavelength is comprised from 815 to 1,400 nm;
the laser power is comprised from 20 mW to 60 W;
the distance from the laser source to the plant is comprised from 10 to 150 cm;
and the amount of plant exposed to the laser is comprised from 0.1 to 1 Kg/m².

2. The process according to claim 1, wherein the process further comprises after parts (c) an additional part to obtain a dry extract, wherein the additional part is selected from:
part (d), which comprises drying the aqueous extract obtained in part (c);
part (e), which comprises:
(i) Precipitating the aqueous extract obtained in part (c) by adding an organic solvent, to obtain a precipitate and a supernatant; and
(ii) Drying the precipitate obtained in part (i); and
part (f), which comprises:
(i) Carrying out one or more extractions of the aqueous extract obtained in part (c) with one or more organic solvents, and separating the aqueous phase and organic phase; and
(ii) drying the aqueous phase obtained in part (i).

3. The process according to claim 2, wherein the drying part is carried out by a method selected from concentration at low pressure, lyophilisation or a combination thereof.

4. The process according to claim 1, wherein the process further comprises a part prior to part (a) of crushing or shredding the plant.

5. The process according to claim 1, wherein in part (a) the weight/volume ratio between the weight of the plant and the water volume is comprised between 1:1 and 1:25.

6. The process according to claim 5, wherein in part (a) the ratio between the weight of the plant obtained in part (a) and the water volume is comprised between 1:5 and 1:15.

7. The process according to claim 1, wherein part (b) is carried out for a period of time comprised from 1 to 200 hours.

8. The process according to claim 1, wherein part (b) is carried out at a temperature comprised from 1 to 90° C.

9. The process according to claim 1, wherein the plant belongs to one or more families selected from the group consisting of Asteraceae, Rosaceae, Lamiaceae, Santalaceae, Crucifrae, Labiatae, Equisetaceae, Saxifraganceae, Compositae, Araliceae, Umbeliferae and mixtures thereof.

10. The process according to claim 1, wherein the plant is Calendula officinalis.

11. The process according to claim 1, wherein the quantity of laser energy is comprised from 0.3 to 3 J/cm².

12. The process according to claim 1, wherein the wavelength is comprised from 830 to 980 nm.

13. The process according to claim 1, wherein the laser power is comprised from 50 mw to 20 W.

14. The process according to claim 1, wherein part (b) is carried out: at a temperature comprised from 70 to 90° C. for a period of time comprised from 1 to 2 hours; or alternatively at a temperature comprised from 3 to 10° C. for a period of time comprised from 100 to 200 hours.

15. An extract obtained by the process as defined in claim 1.

16. A cosmetic or pharmaceutical topical composition comprising an effective amount of the extract as defined in claim 15, together with one or more appropriate topical pharmaceutically or cosmetically acceptable excipients or carriers.

17. A cosmetic method for skin care of a mammal, said method comprising administering to said mammal a topical cosmetic composition as defined in claim 16, wherein the skin care comprises ameliorating one or more of the following symptoms: roughness, desquamation, itching, redness, swelling, dryness and skin pruritus.

18. A method for one or both prophylaxis and treatment of a disease or condition of an inflammatory skin disease or condition comprising
administering an effective amount of the extract defined in claim 16 to a mammal in need of such treatment, together with one or more appropriate topical pharmaceutically acceptable excipients or carriers.

19. The method according to claim 18, wherein the inflammatory skin condition or disease is psoriasis.
